(19) United States
(12) Patent Application Publication
(10) Pub. No.: US 2026/0256512 A1
(43) Pub. Date: Sep. 3, 2026

(54) RESECTOSCOPE VALVE PART FOR A MANUALLY ACTUATABLE WORKING ELEMENT OF A RESECTOSCOPE

(71) Applicant: KARL STORZ SE & Co. KG, Tuttlingen (DE)

(72) Inventors: Rainer HERMLE, Tuttlingen (DE); Judith PÖNISCH, Tuttlingen (DE); Aline ROTH, Tuttlingen (DE); Uwe WITTKE, Tuttlingen (DE)

(21) Appl. No.: 18/865,798

(22) PCT Filed: May 15, 2023
(86) PCT No.: PCT/EP2023/063026

§ 371 Date: Nov. 14, 2024

(30) Foreign Application Priority Data
May 17, 2022 (DE) 10 2022 112 284.0

(51) Int. Cl. A61B 18/14; A61B 17/00; A61B 17/34

(52) U.S. Cl. CPC A61B 18/149; A61B 2017/00367; A61B 2017/00424; A61B 17/3498

(57) ABSTRACT

A resectoscope valve part for a manually operable working element of a resectoscope. The resectoscope valve part includes a substantially cylindrical valve body which is configured to be insertable into a valve seat along a longitudinal axis, a base portion arranged at the end of the valve body, an actuating portion connected to the base portion, and a sealing element arranged on the valve body. The sealing element is configured to axially secure the valve body in the valve seat. Furthermore, a valve arrangement and a resectoscope are provided with the resectoscope valve part.

### CROSS REFERENCE TO RELATED APPLICATIONS

This application is a United States National Phase Application of International Application PCT/EP2023/063026, filed May 15, 2023, and claims the benefit of priority under 35 U.S.C. §119 of German Application 10 2022 112 284.0, filed May 17, 2022, the entire contents of which are incorporated herein by reference.

### FIELD OF THE INVENTION

The present invention relates to a resectoscope valve part for a manually operable working element of a resectoscope. Furthermore, the invention relates to a valve arrangement and to a resectoscope.

### TECHNICAL BACKGROUND

Surgical instruments are used for different applications. For example, they can be used in the area of minimally invasive surgery and have a surgical tool. Surgical instruments can be configured as resectoscopes and have a working element that is configured, for example, to guide the surgical tool, such as an electrode.

Resectoscopes are particularly well known for urological or, for example, gynecological applications. The working element with the electrode can be inserted into the patient's body through the urethra to a surgical site. At one distal end (i.e., remote from the user), for example, the electrode has a cutting loop that can be used to ablate tissue. The ablated tissue can be removed again through a drain channel by means of an irrigation liquid that is guided through an irrigation channel or inflow channel to the surgical site. The inflow and drain channels enable continuous rinsing, which also serves to keep a viewing window clear to ensure a perfect endoscopic view at all times.

Surgical instruments generally have a handle with a slide that is mounted such that it can be moved along a longitudinal axis of the surgical instrument and is connected to the surgical tool. If an electrode is inserted into the working element, the cutting loop of the electrode can be moved in the axial direction by moving the slide axially, for example to ablate tissue.

In order to transport the irrigation liquid and ablated material from the irrigation channel or to supply irrigation liquid, at least one valve is arranged in a region near the handle. In particular, one valve is connected to the inlet channel and one valve is connected to the outlet channel. It is also conceivable that a valve is connected to both channels and can either supply irrigation liquid or remove material with irrigation liquid.

A surgical instrument with a manually operable working element is thus known from DE 10 2019 112 594 A1. A valve arrangement with a valve part can be operated via a lever arranged on it so that the valve can either be opened or closed.

The surgical instrument is usually an instrument that can be operated with one hand. The valve can therefore be operated via the lever from either side of the valve, i.e. from a position to the right or left of the surgical instrument.

The lever allows the instrument to be configured for either right-handed or left-handed use.

Furthermore, the valve part can be detachable from the surgical instrument. For example, the valve arrangement can be configured as a type of tap and plug unit, wherein the valve part can be removed, disassembled and cleaned. The valve part can then be reused.

### SUMMARY OF THE INVENTION

Against this background, the present invention is based on the object of providing an improved valve part or an improved valve arrangement.

According to the invention, this object is achieved by a resectoscope valve part having the features of claim 1, by a valve arrangement having the features of claim 13, and by a resectoscope having the features of claim 15.

Accordingly, the following are provided:
A resectoscope valve part for a manually operable working element of a resectoscope, with a substantially cylindrical valve body which is configured to be insertable into a valve seat along a longitudinal axis, a base portion arranged at the end of the valve body, an actuating portion connected to the base portion, and a sealing element arranged on the valve body which is configured to axially secure the valve body in the valve seat.
A valve arrangement comprising a resectoscope valve part and a valve seat for receiving the valve body, the valve seat having on an inner surface a circumferential groove for the locking accommodation of the sealing element.
A resectoscope with a manually operable working element which has a valve arrangement.

According to one aspect of the invention, the recognition underlying the present invention is to provide a valve part that can be replaced cost-effectively.

The idea underlying the present invention with regard to the second aspect is to design the valve part for single use. In particular, this can enable a cost-effective valve part with the possibility of axially securing the valve body in the valve seat. This is achieved in particular by a sealing element that enables a locking connection between the valve part and the valve seat.

Such a valve can also be called a single-use valve. The valve part can, for example, be manufactured quickly and easily as an injection-molded part. In this way, a single use without complicated cleaning processes can offer advantages over multiple-use valves, particularly in terms of cost savings. The sealing element is configured especially for use as a single-use valve and at the same time provides an axial securing of the valve body in the valve seat.

The valve body is to be understood as an element that can be detached from the valve seat and can be exchanged. The valve body is in particular a shut-off body for a valve, preferably a rotary slide valve.

Preferably, the valve part is mounted horizontally in the valve seat in an installed state. Horizontal here refers to a plane relative to the surgical instrument when the surgical element is held in front of the body in a position ready for operation.

The valve part can contain Makrolon, polycarbonate, polyether ether ketone (PEEK), polysulfone (OSU), polyetherimide (PEI), or a mixture of these materials. In particular, the valve part can be made of one of these materials. In a further embodiment, the valve part can contain a steel material or a ceramic material. In the case of a steel material, production can be carried out in particular via a metal powder injection molding process (MIM process), and in the case of a ceramic material via an injection molding process.

The valve body is substantially cylindrical in shape. This means that the valve body has a cross section with a substantially constant diameter, particularly with respect to the longitudinal axis. The valve body is therefore not conical. The valve body is also adapted to the shape of the valve seat. All shapes which are cylindrical, taking into account any manufacturing tolerances, in particular even if they have partial recesses or openings along the longitudinal axis, are substantially cylindrical.

The resectoscope valve part is configured in particular for single use. In another version, it can also be configured for multiple use.

The sealing element arranged in the valve body is preferably configured for sealing in addition to axial securing.

Advantageous embodiments and developments are shown in the further dependent claims and in the description with reference to the figures in the drawing.

When a valve part is mentioned below, this also includes the resectoscope valve part according to the invention.

According to an advantageous embodiment, an actuating portion connected to the base portion can be provided, which has a first actuating extension configured for left-handed operation and a second actuating extension configured for right-handed operation, which are each configured to actuate the valve body with respect to the longitudinal axis.

The actuating portion can have different designs. For example, it can be configured as a straight, flat element.

The base portion allows the actuating portion to be redirected as desired so that it can be reached from both sides of the surgical instrument.

In particular, the valve body can be formed in one piece with the actuating portion and can be made detachable from the valve seat.

The actuating portion is preferably oriented horizontally when the resectoscope valve part is installed in the valve seat.

The actuating portion can have a length of 15 mm to 30 mm, in particular 20 mm to 26 mm, preferably 25 mm. The valve seat is preferably half as long as the actuating portion.

According to an advantageous embodiment, the actuating portion can have a connecting web parallel to the longitudinal axis of the valve body, connecting the first and second actuating extensions, and which is firmly connected, in particular in one piece, to the valve body via the base portion. Preferably, the actuating portion is formed in one piece with the valve body. The base portion can be an end-side portion on the valve body that provides a connection region between the actuating portion and the valve body. In particular, the actuating portion is connected to the valve body only on one side. The base portion is preferably a region that is not inserted into the valve seat.

According to an advantageous embodiment, the actuating portion, in particular the connecting web of the actuating portion, can have a greater axial length than the valve body. In an installed state, the valve part is preferably mounted horizontally in the valve seat; therefore, if the actuating portion runs parallel to the longitudinal axis of the valve body, the actuating portion can be made longer than the valve body and at the same time not hinder the valve body in a rotational movement. The valve part can be inserted into a valve seat from the left or right side; in both cases, right-and left-handed operation is possible.

According to an advantageous embodiment, the actuating portion can be substantially U-shaped, wherein the first and second actuating extensions are each aligned substantially transversely to the longitudinal axis of the valve body. Substantially transversely means that in particular the actuating extensions run at an angle or obliquely with respect to the actuating portion. This can provide a larger contact surface for gripping with the fingers, allowing the valve to be adjusted safely and ergonomically.

According to an advantageous embodiment, the actuating portion can project beyond the valve body at an end opposite the base portion. If the actuating portion, in particular the connecting web, projects beyond the valve body at the end opposite the base portion and is simultaneously arranged on the side of the base portion, the actuating portion, in particular the connecting web, can be reached from both sides of the surgical instrument.

According to an advantageous embodiment, the valve body can have a passage opening which is aligned transversely to the longitudinal axis of the valve body, so that the valve body can be adjusted to an open or closed position by rotating the actuating portion about the longitudinal axis. The rotation can be triggered and/or controlled by the actuating portion. Preferably, the actuating portion moves by the same angle as the through-opening is rotated, or by the same angle as the valve body. This allows an operator to see at what angle the through-opening is, i.e. whether the valve is (partially) open or (partially) closed.

According to an advantageous embodiment, the sealing element for axially securing the valve body can be configured as a clamping seal, preferably as an annular seal, that can be clamped into a mating contour in the valve seat. If the valve part is inserted laterally into the valve seat, the valve part is prevented from moving in the direction of the longitudinal axis of the valve body by the sealing element. As a result, a conical design of the valve body or valve seat can be dispensed with.

According to an advantageous embodiment, the sealing element can engage in a contour, in particular an annular groove, in the valve body. The contour serves as an axial securing, as it holds the sealing element in a desired plane or position relative to the valve body.

According to an advantageous embodiment, an extension can be arranged on the valve body, which extension can be contacted with an outer surface of the valve seat in a sliding manner, in particular under pre-tension, such that a continuous adjustment into different rotational positions of the valve body is possible. Preferably, the extension is configured to be multiple times shorter than a length of the valve body. The extension can be a pin-shaped element arranged parallel to and spaced apart from a surface of the valve body at an end-side region on the valve body. A tension can be built up when the extension comes into contact with the valve seat in the installed state of the valve part.

According to an advantageous embodiment, the extension can be fashioned as a spring element which is arranged protruding in a pin-like manner on the base portion of the valve body and is aligned substantially parallel to the longitudinal axis of the valve body. In particular, the extension is arranged on the base portion at a position opposite the actuating portion. The extension can be formed in one piece with the base portion and/or the valve body. In particular, the extension contains Makrolon, polycarbonate, polyether ether ketone (PEEK), polysulfone (OSU), polyetherimide (PEI), or a mixture of these materials. In particular, the extension can consist of one of these materials. Substantially parallel means that the extension extends in the axial direction of the valve body, taking into account any manufacturing tolerances.

According to an advantageous embodiment, the base portion and the actuating portion can be formed in one piece with one another, preferably from a duromer material. The use of a duromer material enables a hard material that can withstand loading. In a further embodiment, the base portion and the actuating portion can be formed in multiple parts. Materials other than a duromer material are also conceivable in all embodiments.

The sealing element preferably contains a soft material, such as silicone. In particular, the sealing element can be formed from an O-ring made of silicone. For example, Perbunan can be used as a material. It is particularly important that the material has sufficient elasticity so that a sealing can be achieved.

A one-piece valve part also offers advantages over multi-piece solutions, such as a multi-piece tap and plug unit, which must be assembled and requires threaded connections.

Through the shape of the valve body, in particular the cylindrical shape, in combination with the sealing element, a securing of the valve part in the valve seat and a sealing can be achieved at the same time.

According to an advantageous embodiment of the valve arrangement, at least one locking position for the extension can be formed on an outer surface of the valve seat, so that the valve body can be fixed by the extension in locking fashion in a rotational position between an open and a closed position, in particular formed by a stop in each case. In a further embodiment, the stop can also be understood as a locking position.

According to an advantageous embodiment of the valve arrangement, at least two locking positions for the extension can be formed on an outer surface of the valve seat, so that the valve body can be fixed in locking fashion in different rotational positions by the extension. This makes it possible to provide a valve with defined valve positions, for example for a pressure-controlled resectoscope. The spring element allows the valve part to lock into the valve seat in defined positions.

It would also be conceivable for the spring element to be arranged on the valve seat and for defined locking positions to be arranged on the surface of the valve part.

According to a second aspect, the object underlying the invention is achieved by a valve part having the features of embodiment 1, by a valve arrangement having the features of embodiment 12, and by a resectoscope having the features of embodiment 14.

Accordingly, the following are provided:
A valve part for a manually operated work element of a surgical instrument, in particular a resectoscope, with a substantially cylindrical valve body which is configured to be insertable into a valve seat along a longitudinal axis, with a base portion arranged at the end side on the valve body, with a sealing element arranged on the valve body and with an actuating portion connected to the base portion, which actuating portion has a first actuating extension configured for left-handed operation and a second actuating extension configured for right-handed operation, each of which is configured to actuate the valve body in relation to the longitudinal axis.
A valve arrangement having a valve part and a valve seat for receiving the valve body, the valve seat having on an inner surface a circumferential groove for the locking accommodation of the sealing element.
A resectoscope with a manually operable working element which has a valve arrangement.

According to the second aspect, an underlying finding is that right-and left-handed operability are to be equally enabled when closing or opening a valve on a surgical instrument. This is particularly advantageous when it comes to a surgical instrument that is operated with one hand.

The underlying idea regarding the second aspect is to make the lever for operating the valve accessible from both sides of the valve, i.e. from the left and right of the surgical instrument.

In contrast to known valve parts with a lever, the valve part according to the invention can be operated by the actuating portion not only from one side of the valve, i.e. from a position on the right or left of the surgical instrument, but is configured equally for left-handed and right-handed people.

This can be implemented by the actuating portion, which is configured for both left-handed and right-handed operation due to the first and second actuating extensions.

This means that the surgical instrument can be used flexibly by left-handed and right-handed people without any assembly effort.

The actuating portion can have different designs. For example, it can be configured as a straight, flat element.

The base portion allows the actuating portion to be redirected as desired so that it can be reached from both sides of the surgical instrument.

The valve body is to be understood as an element that can be detached from the valve seat and can be exchanged. The valve body is in particular a shut-off body for a valve, preferably a rotary slide valve.

In particular, the valve body can be formed in one piece with the actuating portion and can be made detachable from the valve seat.

Preferably, the valve part is mounted horizontally in the valve seat in an installed state. The actuating portion is also preferably aligned horizontally when the valve part is installed in the valve seat. Horizontal here refers to a plane relative to the surgical instrument when the surgical element is held in front of the body in a position ready for operation.

The valve part can contain Makrolon, polycarbonate, polyether ether ketone (PEEK), polysulfone (OSU), polyetherimide (PEI), or a mixture of these materials. In particular, the valve part can be made of one of these materials. In a further embodiment, the valve part can contain a steel material or a ceramic material. In the case of a steel material, production can be carried out in particular via a metal powder injection molding process (MIM process), and in the case of a ceramic material via an injection molding process.

The actuating portion can have a length of 15 mm to 30 mm, in particular 20 mm to 26 mm, preferably 25 mm. The valve seat is preferably half as long as the actuating portion.

The valve body is substantially cylindrical in shape. This means that the valve body has a cross section with a substantially constant diameter, particularly with respect to the longitudinal axis. The valve body is therefore not conical. The valve body is also adapted to the shape of the valve seat. All shapes which are cylindrical, taking into account any manufacturing tolerances, in particular even if they have partial recesses or openings along the longitudinal axis, are substantially cylindrical.

The valve part is configured in particular for single use. In another version, it can also be configured for multiple use.

The sealing element arranged in the valve body is preferably configured for sealing in addition to axial securing.

According to an advantageous embodiment, the actuating portion can have a connecting web parallel to the longitudinal axis of the valve body, connecting the first and second actuating extensions, and which is firmly connected, in particular in one piece, to the valve body via the base portion. Preferably, the actuating portion is formed in one piece with the valve body. The base portion can be an end-side portion on the valve body that provides a connection region between the actuating portion and the valve body. In particular, the actuating portion is only connected to the valve body on one side. The base portion is preferably a region that is not inserted into the valve seat.

According to an advantageous embodiment, the actuating portion, in particular the connecting web of the actuating portion, can have a greater axial length than the valve body. In an installed state, the valve part is preferably mounted horizontally in the valve seat; therefore, if the actuating portion runs parallel to the longitudinal axis of the valve body, the actuating portion can be made longer than the valve body and at the same time not hinder the valve body in a rotational movement. The valve part can be inserted into a valve seat from the left or right side; in both cases, right-and left-handed operation is possible.

According to an advantageous embodiment, the actuating portion can be substantially U-shaped, wherein the first and second actuating extensions are each aligned substantially transversely to the longitudinal axis of the valve body. Substantially transversely means that in particular the actuating extensions run at an angle or obliquely with respect to the actuating portion. This can provide a larger contact surface for gripping with the fingers, allowing the valve to be adjusted safely and ergonomically.

According to an advantageous embodiment, the actuating portion can project beyond the valve body at an end opposite the base portion. If the actuating portion, in particular the connecting web, projects beyond the valve body at the end opposite the base portion and is simultaneously arranged on the side of the base portion, the actuating portion, in particular the connecting web, can be reached from both sides of the surgical instrument.

According to an advantageous embodiment, the valve body can have a through-opening which is aligned transversely to the longitudinal axis of the valve body, so that the valve body can be adjusted into an open or closed position by rotating the actuating portion about the longitudinal axis. The rotation can be triggered and/or controlled by the actuating portion.

Preferably, the actuating portion moves by the same angle as the through-opening is rotated, or by the same angle as the valve body. This allows an operator to see at what angle the through-opening is, i.e. whether the valve is (partially) open or (partially) closed.

According to an advantageous embodiment, the sealing element can be configured for axially securing the valve body in the valve seat, in particular as a clamping seal that can be clamped into a mating contour in the valve seat, preferably as an annular seal. If the valve part is inserted laterally into the valve seat, the valve part is prevented from moving in the direction of the longitudinal axis of the valve body by the sealing element. As a result, a conical design of the valve body or valve seat can be dispensed with.

According to an advantageous embodiment, the sealing element can engage in a contour, in particular an annular groove, in the valve body. The contour serves as an axial securing, as it holds the sealing element in a desired plane or position relative to the valve body.

According to an advantageous embodiment, an extension can be arranged on the valve body, which extension can be contacted with an outer surface of the valve seat in a sliding manner, in particular under pre-tension, such that a continuous adjustment into different rotational positions of the valve body is possible. Preferably, the extension is configured to be multiple times shorter than a length of the valve body. The extension can be a pin-shaped element arranged parallel to and spaced apart from a surface of the valve body at an end-side region on the valve body. A tension can be built up when the extension comes into contact with the valve seat in the installed state of the valve part.

According to an advantageous embodiment, the extension can be fashioned as a spring element which is arranged protruding in a pin-like manner on the base portion of the valve body and is aligned substantially parallel to the longitudinal axis of the valve body. In particular, the extension is arranged on the base portion at a position opposite the actuating portion. The extension can be formed in one piece with the base portion and/or the valve body. In particular, the extension contains Makrolon, polycarbonate, polyether ether ketone (PEEK), polysulfone (OSU), polyetherimide (PEI), or a mixture of these materials. In particular, the extension can consist of one of these materials. Substantially parallel means that the extension extends in the axial direction of the valve body, taking into account any manufacturing tolerances.

According to an advantageous embodiment, the valve part can be configured for single use; in particular, the valve body, the base portion and the actuating portion can be formed in one piece with one another, preferably from a duromer material. Such a valve can also be called a single-use valve. The valve part can, for example, be manufactured quickly and easily as an injection-molded part. In this way, a single use without complicated cleaning processes can offer advantages over multiple-use valves, particularly in terms of cost savings. The sealing element is configured especially for use as a single-use valve and at the same time provides an axial securing of the valve body in the valve seat. The use of a duromer material enables a hard material that can withstand loading.

In a further embodiment, the base portion and the actuating portion can be formed in multiple parts. Materials other than a duromer material are also conceivable in all embodiments.

The sealing element preferably contains a soft material, such as silicone. In particular, the sealing element can be formed from an O-ring made of silicone. For example, Perbunan can be used as a material. It is particularly important that the material has sufficient elasticity so that a sealing can be achieved.

A one-piece valve part also offers advantages over multi-piece solutions, such as a multi-piece tap and plug unit, which must be assembled and requires threaded connections.

Through the shape of the valve body, in particular the cylindrical shape, in combination with the sealing element, a securing of the valve part in the valve seat and a sealing can be achieved at the same time.

According to an advantageous embodiment of the valve arrangement, at least one locking position for the extension can be formed on an outer surface of the valve seat, so that the valve body can be fixed by the extension in locking fashion in a rotational position between an open and a closed position, in particular formed by a stop in each case. In a further embodiment, the stop can also be understood as a locking position.

According to an advantageous embodiment of the valve arrangement, at least two locking positions for the extension can be formed on an outer surface of the valve seat, so that the valve body can be fixed in locking fashion in different rotational positions by the extension. This makes it possible to provide a valve with defined valve positions, for example for a pressure-controlled resectoscope. The spring element allows the valve part to lock into the valve seat in defined positions.

It would also be conceivable for the spring element to be arranged on the valve seat and for defined locking positions to be arranged on the surface of the valve part.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The present invention is explained in greater detail below with reference to the exemplary embodiments shown in the schematic figures of the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view showing a surgical instrument with a valve arrangement;

FIG. 2 is an exploded perspective view showing a resectoscope valve part according to an embodiment with a valve seat;

FIG. 3 is a perspective view further showing the embodiment of FIG. 2;

FIG. 4 is a plan view of a resectoscope valve part;

FIG. 5 is a detail view from FIG. 3.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures in the drawing, like, functionally like and identically acting elements, features and components are each provided with the same reference signs, unless otherwise specified.

### DESCRIPTION OF EXEMPLARY EMBODIMENTS

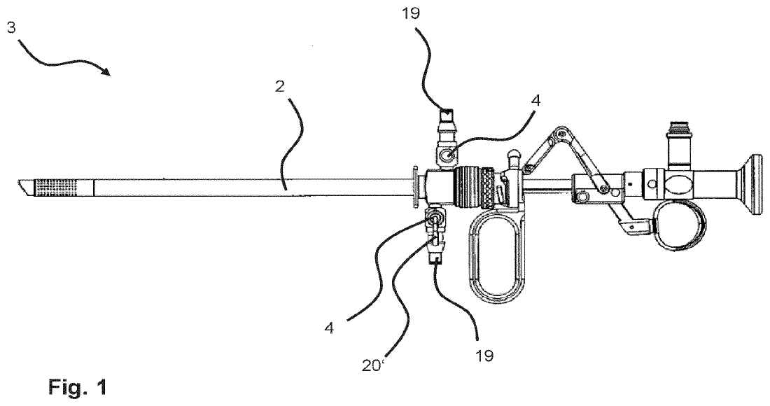

Referring to the drawings, FIG. 1 shows a surgical instrument in the form of a resectoscope 3 with a working element 2. A valve arrangement with two valves is arranged between the working element 2 and a handle. Each of these valves has a valve seat 4, with one valve seat 4 arranged above and one valve seat 4 arranged below the working element. Each of these valves can be connected to an inlet and/or an outlet 19. In the downwardly oriented valve, a valve part with a lever 20′ can be seen in the valve seat 4, as is known in the prior art.

The two valve seats 4 shown in FIG. 1 are also suitable for accommodating a resectoscope valve part 1 according to the invention. A resectoscope valve part 1 according to the invention is also configured to be arranged on other manually operable working elements 2 of surgical instruments.

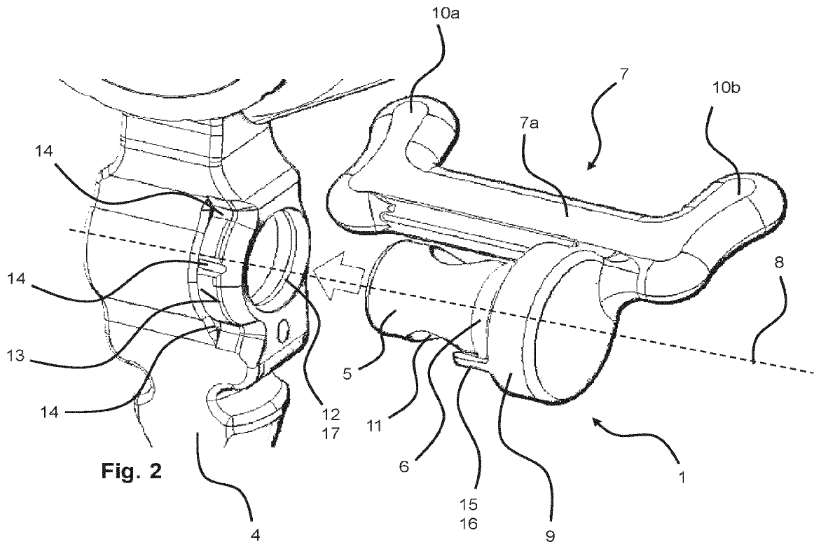

FIG. 2 shows a resectoscope valve part 1 according to an embodiment and a valve seat 4. The resectoscope valve part 1 has a valve body 5, a base portion 9 and an actuating portion 7. With the valve body 5, the resectoscope valve part 1 is inserted horizontally along a longitudinal axis 8 into the valve seat 4. Only one possibility is shown, namely how the resectoscope valve part 1 can be inserted from the right into the valve seat 4. It would also be conceivable for the resectoscope valve part 1 to be rotated by 180° and inserted into the valve seat 4 from the left side in the illustration. In the case of both options, the actuating portion 7 also has the desired function, wherein the valve seat should then be configured mirrored to a vertical axis.

The valve body 5 is substantially cylindrical. A through-opening 11 is arranged in the cylindrical valve body 5 transversely to the longitudinal axis 8. The orientation of the through-opening 11 can be changed by moving, i.e. rotating, the actuating portion 7. This allows the valve to be (partially) opened or (partially) closed.

The base portion 9 is arranged on an end portion of the valve body 5 and is configured as a connection between the actuating portion 7 and the valve body 5. The base portion 9 is not inserted into the valve seat 4, but remains outside in the installed state. A sealing element 6, which is configured as a circumferential annular seal, is arranged directly behind the base portion 9. The sealing element 6 is placed in a contour 18 in the valve body 5. A mating contour 17 is arranged as a circumferential groove 12 in the valve seat 4. In the installed state, this allows an axial securing of the valve body 5 to be achieved with respect to the valve seat 4.

Furthermore, an extension 15 in the form of a spring element 16, which is pin-shaped, is arranged on the base portion 9. The extension 15 is also arranged substantially parallel to the longitudinal axis 8 and spaced apart from the sealing element 6 and the valve body 5. The extension 15 can engage in one of the three locking positions 14 formed in the valve seat 4. The locking positions 14 are arranged on an outer surface 13 of the valve seat 4 such that the resectoscope valve part 1 can be fixed in different rotational positions. This can be achieved by rotating the actuating portion 7 upwards or downwards.

In an embodiment not shown, only one locking position 14 can be provided. This can be arranged centrally between two stops. One of the stops is configured in particular for a closed position, one for an open position of the valve. The stops can also be referred to as locking positions.

In this embodiment, the actuating portion 7 has a connecting web 7a which runs substantially parallel to the longitudinal axis 8. At each end of the connecting web 7a, an actuating extension 10a, 10b is arranged, wherein the actuating extension 10a can be reached from a left position and the actuating extension 10b can be reached from a right position with respect to the surgical instrument. In this embodiment, the actuating portion 7 is configured with a greater axial length than the valve body 5, so that the left position is also easily accessible.

In this embodiment, the resectoscope valve part 1 is formed in one piece and can be manufactured, for example, by an injection molding method. This makes it possible to provide a low-cost resectoscope valve part 1 which is particularly suitable for single use.

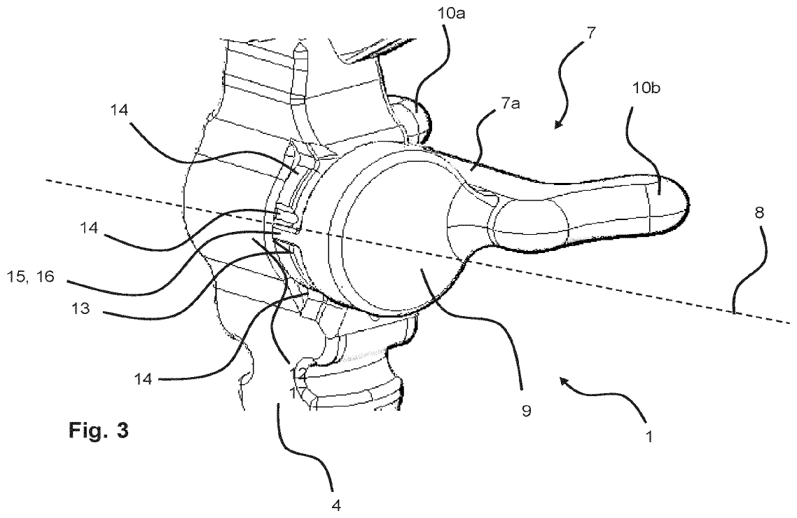

FIG. 3 shows another view of the embodiment of FIG. 2 in a connected state. It can be seen that the base portion 9 is not inserted into the valve seat 4.

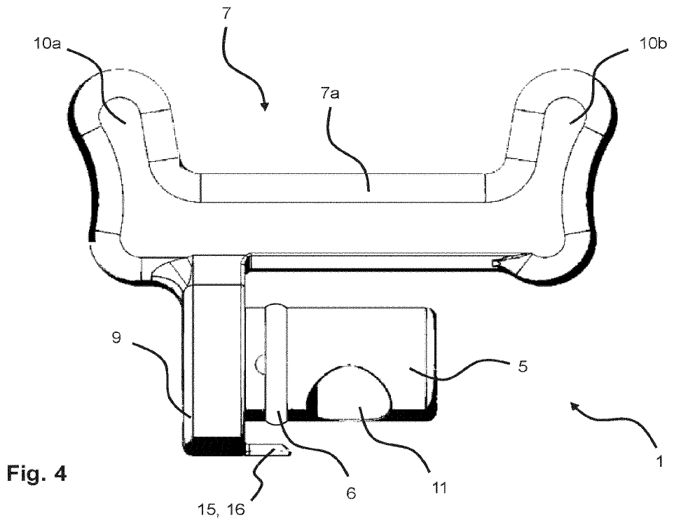

FIG. 4 shows a plan view of a resectoscope valve part 1. The actuating extensions 10a, 10b have in particular a length of 7 to 15 mm, preferably 10 to 12 mm, in particular 11 mm. The two actuating extensions 10a, 10b can be arranged at a distance (here at a horizontal distance) of 15 mm to 25 mm, in particular 20 mm to 22 mm, preferably 21 mm, measured from a central axis of the corresponding actuating extension 10a, 10b.

In FIG. 4, the resectoscope valve part 1 is made in mirrored fashion relative to the embodiment according to FIGS. 2 and 3. In such an embodiment, the valve body 5 can be inserted into a valve seat 4 from the left side. As already shown with regard to the embodiment according to FIGS. 2 and 3, the actuating portion 7 projects beyond the valve body 5 at an end opposite the base portion 9. As a result, the resectoscope valve part 1 is particularly advantageously equally configured for left-handed and right-handed operation.

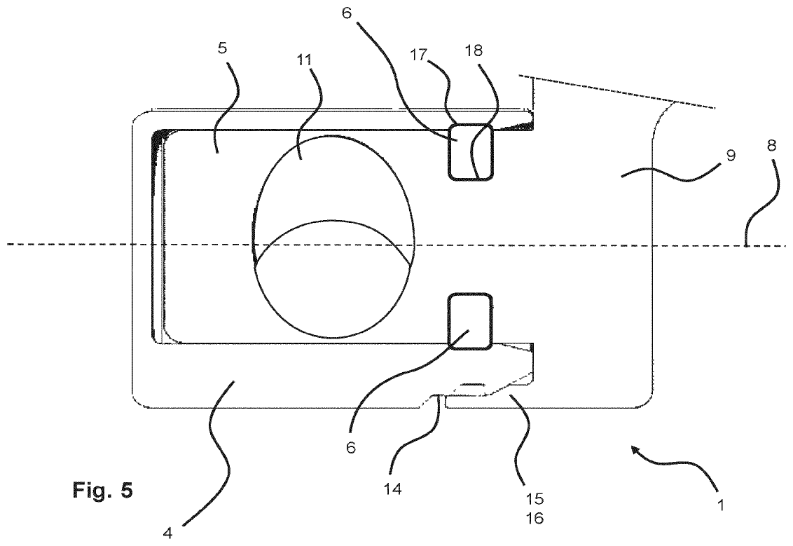

FIG. 5 shows a detail view from 3. In the sectional view, it can be seen that the resectoscope valve part 1 is inserted into the valve seat 4 and the extension 15 engages in one of the three locking positions 14. This can be seen from the fact that the extension 15 is shown partially covered by a portion of the valve seat 4. Furthermore, it can be seen that the sealing element 6 engages in a contour 18 in the valve body 5 and in a mating contour 17 in the valve seat 4. As a result, the complete resectoscope valve part 1 cannot move along the longitudinal axis 8 and is axially fixed within the valve seat 4.

Although the present invention has been fully described above with reference to preferred exemplary embodiments, it is not limited thereto, but can be modified in a variety of ways.

For example, the number of locking positions 14 can vary. Likewise, the locking positions 14 can be arranged at different positions. Furthermore, the actuating portion 7 can be configured differently from the embodiment shown. Thus, the actuating portion 7 does not have to have two actuating extensions 10 aligned substantially transversely to the longitudinal axis 8, and therefore does not have to be U-shaped. To implement the idea of the invention, it is necessary only that the actuating extension 10a can be reached from one side and the actuating extension 10b from the other side of the surgical instrument when this instrument is in use during one-handed operation. In a first aspect, this makes it possible to realize equal operability for both left-and right-handed users.

In general, the extension can enable a positioning of the resectoscope valve part 1 in defined valve positions, through the locking positions 14. This allows the supply or discharge of irrigation liquid to be individually adjusted.

In a further aspect, the resectoscope valve part 1, which can be inserted laterally into the valve seat 4, is held axially in the desired position by the sealing element 6. This ensures an axial securing of the resectoscope valve part 1. This is also achieved by the fact that the valve body 5 is cylindrical in shape. In particular, this makes it possible to implement a resectoscope valve part 1 for single use, a so-called single-use valve, which can be easily assembled and disassembled. Due to the one-piece design, which can be achieved for example through an injection molding process, a low-cost resectoscope valve part 1 can be provided which is suitable for single use, has no threaded elements, requires no assembly effort, and does not need to be expensively cleaned.

Further embodiments can be configured according to a second aspect as follows.

The previously explained figure descriptions apply in the same way to the following embodiments, wherein the resectoscope valve part 1 is configured as a valve part 1′, and wherein the valve part is configured for a manually operable working element 2 of a surgical instrument.

The object underlying the invention can therefore also be achieved by a valve part 1′ with the following features. The reference numeral 1′ designates a general valve part, where reference is made not specifically to a resectoscope valve part 1, i.e. to a valve part specifically for a resectoscope, but to various types of valves, in particular for a wide variety of surgical instruments.

In one embodiment, a valve part 1′ for a manually operable working element 2 of a surgical instrument, in particular a resectoscope 3, can be configured accordingly, having: a substantially cylindrical valve body 5 which is configured to be insertable into a valve seat 4 along a longitudinal axis 8; a base portion 9 arranged at the end side on the valve body 5; a sealing element 6 arranged on the valve body 5; and an actuating portion 7 connected to the base portion 9 and having a first actuating extension 10a configured for left-handed operation and a second actuating extension 10b configured for right-handed operation, each of which is configured to actuate the valve body 5 in relation to the longitudinal axis 8.

The actuating portion can have different designs. For example, it can be configured as a straight, flat element.

The base portion allows the actuating portion to be redirected as desired so that it can be reached from both sides of the surgical instrument.

In a further embodiment of the valve part 1′, the actuating portion 7 can have a connecting web 7a which runs parallel to the longitudinal axis 8 of the valve body 5 and connects the first and second actuating extensions 10a, 10b and is firmly connected, in particular in one piece, to the valve body 5 via the base portion 9.

In a further embodiment of the valve part 1′, the actuating portion 7, in particular the connecting web 7a of the actuating portion 7, can have a greater axial length than the valve body 5.

In a further embodiment of the valve part 1′, the actuating portion 7 can be substantially U-shaped, wherein the first and second actuating extensions 10a, 10b are each aligned substantially transversely to the longitudinal axis 8 of the valve body 5.

In a further embodiment of the valve part 1′, the actuating portion 7 can protrude beyond the valve body 5 at an end opposite the base portion 9.

In a further embodiment of the valve part 1′, the valve body 5 can have a through opening 11 which is aligned transversely to the longitudinal axis 8 of the valve body 5, so that the valve body can be adjusted into an open or closed position by rotating the actuating portion 7 about the longitudinal axis 8.

In a further embodiment of the valve part 1′, the sealing element 6 can be configured to axially secure the valve body 5 in the valve seat 4, in particular as a clamping seal that can be clamped into a mating contour 17 in the valve seat 4, preferably as an annular seal.

In a further embodiment of the valve part 1′, the sealing element 6 can engage in a contour 18, in particular an annular groove, in the valve body 5.

In a further embodiment of the valve part 1′, an extension 15 can be arranged on the valve body 5, which extension can be contacted with an outer surface of the valve seat 4 in a sliding manner, in particular under pre-tension, such that a continuous adjustment into different rotational positions of the valve body is possible.

In a further embodiment of the valve part 1′, the extension 15 can be configured as a spring element 16 which is arranged protruding in a pin-like manner from the base portion 9 of the valve body 5 and is aligned substantially parallel to the longitudinal axis 8 of the valve body 5.

In a further embodiment of the valve part 1′, the valve part 1 can be configured for single use, in particular the valve body 5, the base portion 9 and the actuating portion 7 are formed in one piece with one another, preferably from a duromer material.

Such a valve can also be called a single-use valve. The valve part can, for example, be manufactured quickly and easily as an injection-molded part. In this way, a single use without complicated cleaning processes can offer advantages over multiple-use valves, particularly in terms of cost savings. The sealing element is configured especially for use as a single-use valve and at the same time provides an axial securing of the valve body in the valve seat.

Another embodiment shows a valve arrangement, having a valve part 1 according to one of the preceding embodiments, and a valve seat 4 for receiving the valve body 5, wherein the valve seat 4 has a circumferential groove 12 on an inner surface for the locking accommodation of the sealing element 6.

In a further embodiment of the valve arrangement, with a valve part, at least two locking positions 14 for the extension 15 can be formed on an outer surface 13 of the valve seat 4, so that the valve body 5 can be fixed in locking fashion in different rotational positions by the extension 15.

A further embodiment relates to a resectoscope 3 with a manually operable working element 2, which has a valve arrangement as described above.

Accordingly, the following are provided as further embodiments:
1. A valve part (1) for a manually operable working element (2) of a surgical instrument, in particular a resectoscope (3), comprising:
   a substantially cylindrical valve body (5) which is configured to be insertable into a valve seat (4) along a longitudinal axis (8);
   a base portion (9) arranged at the end side of the valve body (5);
   a sealing element (6) arranged on the valve body (5); and
   an actuating portion (7) connected to the base portion (9) and having a first actuating extension (10a) configured for left-handed operation and a second actuating extension (10b) configured for right-handed operation, each of which is configured to actuate the valve body (5) in relation to the longitudinal axis (8).
2. The valve part (1) according to embodiment 1, characterized in that the actuating portion (7) has a connecting web (7a) which runs parallel to the longitudinal axis (8) of the valve body (5), connects the first and second actuating extensions (10a, 10b) and is firmly connected, in particular in one piece, to the valve body (5) via the base portion (9).
3. The valve part (1) according to embodiment 1 or 2, characterized in that the actuating portion (7), in particular the connecting web (7a) of the actuating portion (7), has a greater axial length than the valve body (5)
4. The valve part (1) according to any one of the preceding embodiments, characterized in that the actuating portion (7) is substantially U-shaped, wherein the first and second actuating extensions (10a, 10b) are each aligned substantially transversely to the longitudinal axis (8) of the valve body (5).
5. The valve part (1) according to any one of the preceding embodiments, characterized in that the actuating portion (7) projects beyond the valve body (5) at an end opposite the base portion (9).
6. The valve part (1) according to any one of the preceding embodiments, characterized in that the valve body (5) has a through-opening (11) which is aligned transversely to the longitudinal axis (8) of the valve body (5), so that the valve body can be adjusted into an open or closed position by rotating the actuating portion (7) about the longitudinal axis (8).
7. The valve part (1) according to any one of the preceding embodiments, characterized in that the sealing element (6) is configured for axially securing the valve body (5) in the valve seat (4), in particular as a clamping seal that can be clamped into a mating contour (17) in the valve seat (4), preferably as an annular seal.
8. The valve part (1) according to any one of the preceding embodiments, characterized in that the sealing element (6) engages in a contour (18), in particular an annular groove, in the valve body (5).
9. The valve part (1) according to any one of the preceding embodiments, characterized in that an extension (15) is arranged on the valve body (5), which extension can be contacted with an outer surface of the valve seat (4) in a sliding manner, in particular under pre-tension, such that a continuous adjustment into different rotational positions of the valve body (1) is possible.
10. The valve part (1) according to embodiment 9, characterized in that the extension (15) is configured as a spring element (16) which is arranged protruding in a pin-like manner on the base portion (9) of the valve body (5) and is aligned substantially parallel to the longitudinal axis (8) of the valve body (5).
11. The valve part (1) according to any one of the preceding embodiments, characterized in that the valve part (1) is configured for a single use; in particular the valve body (5), the base portion (9), and the actuating portion (7) are formed in one piece with one another, preferably from a duromer material.
12. A valve arrangement, with
   a valve part (1) according to any one of the preceding embodiments,
   a valve seat (4) for receiving the valve body (5), wherein the valve seat (4) has a circumferential groove (12) on an inner surface for the locking accommodation of the sealing element (6).
13. The valve arrangement according to embodiment 12, with a valve part according to any one of the embodiments 9 to 11, characterized in that at least two locking positions (14) for the extension (15) are formed on an outer surface (13) of the valve seat (4), so that the valve body (5) can be fixed in different rotational positions by the extension (15).
14. A resectoscope (3) comprising a manually operable working element (2) which has a valve arrangement according to any one of the embodiments 12 or 13.

While specific embodiments of the invention have been shown and described in detail to illustrate the application of the principles of the invention, it will be understood that the invention may be embodied otherwise without departing from such principles.

### LIST OF REFERENCE CHARACTERS

1 resectoscope valve part
2 working element
3 resectoscope
4 valve seat
5 valve body
6 sealing element
7 actuating portion
7a connecting web
8 longitudinal axis
9 base portion
10 actuating extension
11 passage opening
12 circumferential groove
13 outer surface
14 locking position
15 extension
16 spring element
17 mating contour
18 contour
19 inlet/outlet

1. A resectoscope valve part for a manually operable working element of a resectoscope, the resectoscope valve part comprising:
a substantially cylindrical valve body which is configured to be inserted into a valve seat along a longitudinal axis;
a base portion arranged at the end of the valve body;
an actuating portion connected to the base portion; and
a sealing element arranged on the valve body which sealing element is configured to axially secure the valve body in the valve seat.

2. The resectoscope valve part according to claim 1, wherein the actuating portion has a first actuating extension configured for left-handed operation and a second actuating extension configured for right-handed operation, which are each configured to actuate the valve body in relation to the longitudinal axis.

3. The resectoscope valve part according to claim 2, wherein the actuating portion has a connecting web which runs parallel to the longitudinal axis of the valve body, connects the first and second actuating extensions and is firmly connected, in one piece, to the valve body via the base portion.

4. The resectoscope valve part according to claim 1, wherein the actuating portion, has a greater axial length than the valve body.

5. The resectoscope valve part according to claim 1, wherein the actuating portion is substantially U-shaped, wherein the first and second actuating extensions are each aligned substantially transversely to the longitudinal axis of the valve body.

6. The resectoscope valve part according to claim 1, wherein the actuating portion projects beyond the valve body at an end opposite the base portion.

7. The resectoscope valve part according to claim 1, wherein the valve body has a through-opening which is aligned transversely to the longitudinal axis of the valve body, so that the valve body can be adjusted into an open or closed position by rotating the actuating portion about the longitudinal axis.

8. The resectoscope valve part according to claim 1, wherein the sealing element for axially securing the valve body is configured as a clamping seal, comprising an annular seal, which can be clamped into a mating contour in the valve seat.

9. The resectoscope valve part according to claim 1, wherein the sealing element engages in a contour, comprising an annular groove, in the valve body.

10. The resectoscope valve part according to claim 1, wherein an extension is arranged on the valve body, which extension is configured to make sliding contact with an outer surface of the valve seat such that a continuous adjustment into different rotational positions of the valve body is possible.

11. The resectoscope valve part according to claim 10, wherein the extension is configured as a spring element which is arranged protruding in a pin-like configuration on the base portion of the valve body and is aligned substantially parallel to the longitudinal axis of the valve body.

12. The resectoscope valve part according to claim 1, wherein the valve body and the base portion, are formed in one piece with one another.

13. A valve arrangement, comprising:
a resectoscope valve part, the resectoscope valve part comprising:
a substantially cylindrical valve body which;
a base portion arranged at the end of the valve body;
an actuating portion connected to the base portion; and
a sealing element arranged on the valve body which sealing element is configured to axially secure the valve body in the valve seat; and
a valve seat for receiving the valve body, wherein the valve body is configured to be inserted into the valve seat along a longitudinal axis, wherein the valve seat has a circumferential groove on an inner surface configured for a locking accommodation of the sealing element.

14. The valve arrangement according to claim 13,
wherein an extension is arranged on the valve body, which extension is configured to make sliding contact with an outer surface of the valve seat,
wherein at least two locking positions for the extension are formed on the outer surface of the valve seat, so that the valve body can be fixed in different rotational positions by the extension.

15. A resectoscope, comprising a manually operable working element which comprises a valve arrangement, the valve arrangement, comprising:
a resectoscope valve part, the resectoscope valve part comprising:
a substantially cylindrical valve body which;
a base portion arranged at the end of the valve body;
an actuating portion connected to the base portion; and
a sealing element arranged on the valve body which sealing element is configured to axially secure the valve body in the valve seat; and
a valve seat for receiving the valve body, wherein the valve body is configured to be inserted into the valve seat along a longitudinal axis, wherein the valve seat has a circumferential groove on an inner surface configured for a locking accommodation of the sealing element.

16. The valve arrangement according to claim 13, wherein the actuating portion comprises a first actuating extension configured for left-handed operation and a second actuating extension configured for right-handed operation, which first actuating extension and second actuating extension are each configured to actuate the valve body in relation to the longitudinal axis.

17. The valve arrangement according to claim 16, wherein the actuating portion further comprises a connecting web which extends parallel to the longitudinal axis of the valve body, connects the first and second actuating extensions and is fixedly connected, in one piece, to the valve body via the base portion.

18. The valve arrangement according to claim 17, wherein the connecting web of the actuating portion has a greater axial length than the valve body and the actuating portion projects from a location adjacent to the base portion, in a direction away from the base portion, to a location axially beyond an axial extent of the valve body.

19. The valve arrangement according to claim 13, wherein the valve body has a through-opening which is aligned transversely to the longitudinal axis of the valve body, so that the valve body can be adjusted into an open or closed position by rotating the actuating portion about the longitudinal axis.

20. The valve arrangement according to claim 13,
wherein the sealing element is configured as a clamping seal, comprising an annular seal, which is configured to be clamped into a mating contour of the circumferential groove of the valve seat, and
wherein the sealing element engages in a contour, comprising an annular groove, in the valve body.
